(19) United States
(12) Patent Application Publication
(10) Pub. No.: US 2008/0145902 A1
(43) Pub. Date: Jun. 19, 2008

(54) METHOD FOR PREPARATION OF POLYUNSATURATED FATTY ACID-CONTAINING PHOSPHATIDYLSERINE

(76) Inventors: Su Chen, Aliso Viejo, CA (US); Hung Kwong, Aliso Viejo, CA (US)

Correspondence Address: LAW OFFICE OF MICHAEL A. BLAKE, 112 BROAD STREET, MILFORD, CT, 06460, US

(21) Appl. No.: 12/032,164

(22) Filed: Feb. 15, 2008

Related U.S. Application Data
(63) Continuation of application No. 10/762,657, filed on Jan. 21, 2004.

(51) Int. Cl. C07F 9/02; A61K 31/685; C12P 13/00

(52) U.S. Cl. 435/128; 514/78; 554/78

(57) ABSTRACT

A method for the preparation of highly polyunsaturated fatty acid-containing phosphatidylserine and phosphatidic acid by phospholipase D-catalyzed transphosphatidylation of fish liver phosphatidylcholine and L-serine is disclosed.

### CROSS-REFERENCES

This application is a continuation application of U.S. Ser. No. 10/762,657, filed Jan. 21, 2004, to Su Chen, entitled “Preparation of Highly Polyunsaturated Fatty Acid-Containing Phosphatidylserine and Phosphatic Acid”, the contents of which are incorporated by reference herein in their entirety.

### FIELD OF THE INVENTION

The present invention relates to the preparation of highly polyunsaturated fatty acid-containing phosphatidylserine and phosphatidic acid.

### BACKGROUND OF THE INVENTION

Phosphatidylserine and phosphatidic acid are two naturally occurring phospholipid classes. Biochemical and biophysical functions of the phospholipids are well documented and appear to be determined by the composition of phospholipid fatty acid chains. Fatty acid chains with more than two double bonds are generally called highly polyunsaturated fatty acids. Laboratory experiments have shown pharmacological effects of highly polyunsaturated fatty acid-containing phosphatidylserine and phosphatidic acid molecules on enhancing cholinergic neurotransmission.

Due to the difficulty of chemically synthesizing highly polyunsaturated fatty acid-containing phosphatidylserine and phosphatidic acid molecules, chemical extraction and purification of such molecules from bovine brain, particularly the highly polyunsaturated fatty acid-containing phosphatidylserine molecules, is generally practical approach to obtain them. Unfortunately, the risk of bovine spongiform encephalopathy made the use of phosphatidylserine molecules extracted from bovine brain potentially dangerous, and the development of an alternative method to prepare highly polyunsaturated fatty acid-containing phosphatidylserine and phosphatidic acid molecules is desired and lacking. In recent years, new features of phosphatidylserine molecules have been made by phospholipase D-catalyzed transphosphatidylation of egg and soybean phosphatidylcholine and have been used as brain cell nutrients as well. But highly polyunsaturated fatty acid-containing phosphatidylserine and phosphatidic acid molecules are generally lacked in final products.

Fish liver phospholipids contain more than 65% of highly unsaturated fatty acid-containing phosphatidylcholine molecules (less than 5% of phosphatidylserine+phosphatidic acid), and this natural material is considered to be safe for the preparation of highly polyunsaturated fatty acid-containing phosphatidylserine and phosphatidic acid molecules by the phospholipase D-catalyzed transphosphatidylation procedure. Natural phosphatidylcholine can also be readily separated and purified from other phospholipids using chromatographic techniques.

### SUMMARY OF THE INVENTION

The present invention is the preparation of highly polyunsaturated fatty acid-containing phosphatidylserine and phosphatidic acid by phospholipase D-catalyzed transphosphatidylation of Fish Liver Phosphatidylcholine.

### DETAILED DESCRIPTION OF THE INVENTION

The following description and figures are meant to be illustrative only and not limiting. Other embodiments of this invention will be apparent to those of ordinary skill in the art in view of this description.

The following materials are used in the transphosphatidylation procedure:

a: Phospholipase D

Phospholipase D is an enzyme and is commercially available (Sigma Chemical Company; S. Louis, Mo.). Phospholipase D can catalyze the transfer of phosphatidyl group from phosphatylcholine to various primary alcohols.

b: L-Serine

L-Serine is a common amino acid and is commercially available as well (Sigma Chemical Company; S. Louis, Mo.). The chemical structure of L-Serine is:

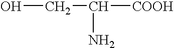

c: Fish Liver Phosphatidylcholine

Phosphatidylcholine (Lecithin) is a naturally occurring phospholipid class. Fish liver is enriched with highly polyunsaturated fatty acid-containing phosphatidylcholine molecules. The structural characterization of these molecules is mainly due to (i) a phosphocholine moiety linked to the sn-3 position of the glycerol backbone; (ii) a variety of diacyl fatty acid chains esterified to the sn-1 and sn-2 positions of the glycerol backbone, and (iii) location of double bond(s) (between 1-6) within unsaturated fatty acid chains with a number of carbon atoms (between 14-22). Fish liver phosphatidylcholine class consists of more than 10 phosphatidylcholine molecules, and a fish liver phosphatidylcholine molecule contains one of any fatty acid chains, which is esterified at sn-1 position of the glycerol backbone, and another one of any fatty acid which is esterified at sn-2 position of the glycerol backbone. The chemical characterization of fish liver phosphatidylcholine species is:

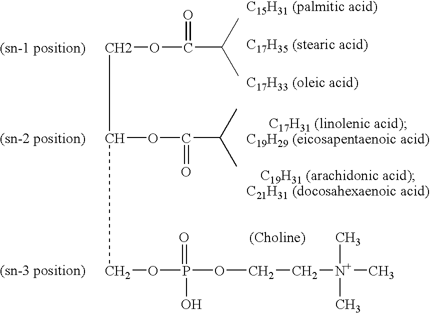

Reference: Lipid Nomenclature, Lipids, Vol. 12, 455-468 (1977))

Laboratory experiments have shown that these highly polyunsaturated fatty acid chains in phospholipid molecules are usually esterified at sn-2 position of the glycerol backbone. Of the phosphatidylcholine molecules from fish liver, the fatty acid chains esterified at the sn-1 position of the glycerol backbone are different, usually including palmitic acid (C.sub.16H.sub.31O.sub.2; containing none of double bond); stearic acid (Cl.sub.8H.sub.35O.sub.2; containing none of double bond); and oleic acid (Cl.sub.8H.sub.33O.sub.2; containing one double bone). The fatty acid chains esterified at the sn-2 position of the glycerol backbone are different too, usually including linolenic acid (C.sub.18H.sub.31O.sub.2; containing two double bonds); arachidonic acid (C.sub.20H.sub.31O.sub.2; containing four double bonds), eicosapentaenoic acid (C.sub.20H.sub.29O.sub.2; containing five double bonds); and docosahexaenoic acid (C.sub.22H.sub.31O.sub.2; containing six double bonds)(See Reference: Su Chen and M. Claeys, J. Agr. Food Chem. Vol. 44, 2416-2423 (1996)).

Phospholipase D-Catalyzed Transphosphatidylation of Phosphatidylcholine (P. Comfurius and R. F. A. Zwaal, Biochim. Biophys. Acta, Vol. 488, p 36-42 (1977)).

At the presence of a L-Serine, a choline moiety within phosphatidylcholine can be replaced by a L-Serine, with phospholipase D-catalyzed transphosphatidylation of phosphatidylcholine, to form phosphatidylserine by this one-step procedure, and phosphatidic acid is also produced as a side product in final products. After the transphosphatidylation, the fatty acid chains esterified at sn-1 and sn-2 positions of the glycerol backbone within final products phosphatidylserine and phosphatidic acid molecules are almost identical to those within phosphatidylcholine precursors used.

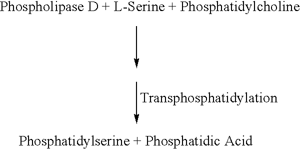

See Reference: P. Comfurius and R. F. Zwaal, Biochim. Biophys. Acta. Vol. 488, p 36-42 (1977))

Characterization of the phosphatidylserine and phosphatidic acid obtained by phospholipase D-catalyzed transphosphatidylation of fish liver phosphatidylcholine

a: Chemistry

After the replacement of a choline moiety at the sn-3 position of the glycerol backbone with an L-serine by phospholipase D-catalyzed transphosphatidylation of fish liver phosphatidylcholine, final products are phosphatidylserine and phosphatidic acid.

The fatty acid chains esterified at sn-1 and sn-2 positions of final products phosphatidylserine and phosphatidic acid molecules are almost identical to those within fish liver phosphatidylcholine precursors after the transphosphatidylation.

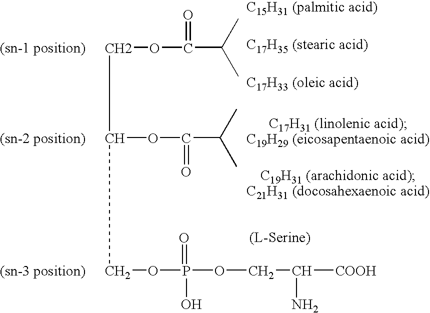

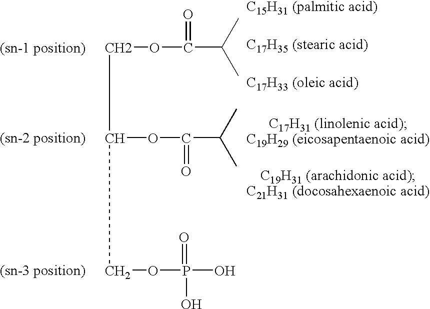

Laboratory experiments have shown that highly polyunsaturated fatty acid-containing phosphatidylserine and phosphatidic acid molecules are more effective as brain cell nutrients on enhancing cholinergic neurotransmission.

The advantages of the present invention are the production of highly polyunsaturated fatty acid-containing phosphatidylserine and phosphatidic acid molecules, which are made by phospholipase D-catalyzed transphosphatidylation of fish live phosphatidylcholine, are much safer when they are used as brain cell nutrients, without the risk of bovine spongiform encephalopathy. Further, choosing fish liver phosphatidylcholine as a precursor to prepare highly polyunsaturated fatty acid-containing phosphatidylserine and phosphatidic acid molecules by the phospholipase D-catalyzed transphosphatidylation procedure is more economic with potentially industrial preparation, compared with small sizes of materials to be used as precursors, such as fish brain and squid skin phosphatidylcholine molecules that also contain highly polyunsaturated fatty acid chains.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A method for the preparation of polyunsaturated fatty acid-containing phosphatidylserine, the method comprising:
combining an L-Serine with a fish liver phosphatidylcholine;
phospholipase D-catalyzed transphosphatidylating the L-Serine and the fish liver phosphatidylcholine; and
producing a polyunsaturated fatty acid-containing phosphatidylserine from the transphosphatidylating step.

2. A method for the preparation of polyunsaturated fatty acid-containing phosphatidylserine, the method comprising:
combining an L-Serine with purified phosphatidylcholine from fish livers;
phospholipase D-catalyzed transphosphatidylating of the L-Serine and fish liver phosphatidylcholine;
producing polyunsaturated fatty acid-containing phosphatidylserine from the phospholipase D-catalyzed transphosphatidylating step.

3. A method for the preparation of polyunsaturated fatty acid-containing phosphatidylserine, the method comprising:
combining L-Serine with fish liver phosphatidylcholine containing fish liver crude phospholipids;
phospholipase D-catalyzed transphosphatidylating the phosphotidylcholine and L-Serine;
producing a polyunsaturated fatty acid-containing phosphatidylserine from the phospholipase D-catalyzed transphosphatidylating step.

4. A method of preparing phosphatidylserine species, the method comprising:
phospholipase D-catalyzed transphosphatidylating of L-Serine and fish liver phosphatidylcholine to produce a phosphatidylserine species as shown in Formula 1: 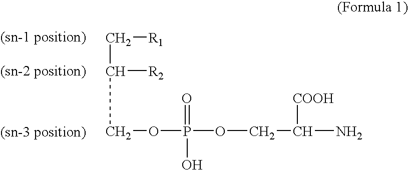
wherein R₁ is a mixture of acyl fatty chains, linked to the sn-1 position, selected from the group consisting of COOC₁₅H₃₁ (acyl fatty chain; palmitic acid; 16:0), COOC₁₇H₃₅ (acyl fatty chain; stearic acid; 18:0), and COOC₁₇H₃₃ (acyl fatty chain; oleic acid; 18:1); and
wherein R₂ is a mixture of acyl fatty chains, linked to the sn-2 position, selected from the group consisting of COOC₁₇H₃₃ (acyl fatty chain; oleic acid; 18:1), COOC₁₇H₃₁ (acyl fatty chain, Linoleic acid; 18:2), COOC₁₉H₃₁ (acyl fatty chain; arachidonic acid; 20:4), COOC₁₉H₂₉ (acyl fatty chain; eicosapentaenoic acid; 20:5 (ω-3)), COOC₂₁H₃₃ (acyl fatty chain; docosapentanoic acid; 22:5), COOC₂₁H₃₁ (acyl fatty chain; docosahexaenoic acid; 22:6 (ω-3)).
